(19) United States
(12) Patent Application Publication
(10) Pub. No.: US 2022/0168133 A1
(43) Pub. Date: Jun. 2, 2022

(54) ANTI-SNORING DEVICE

(71) Applicant: AIRLAB CO., LTD., Jeonju-si Jeollabuk-do (KR)

(72) Inventor: Hyo Kwang LEE, Iksan-si Jeollabuk-do (KR)

(21) Appl. No.: 17/601,269

(22) PCT Filed: Apr. 13, 2020
(86) PCT No.: PCT/KR2020/004960

§ 371 Date: Oct. 4, 2021

(30) Foreign Application Priority Data
Apr. 12, 2019 (KR) 10-2019-0043005

(51) Int. Cl. A61F 5/56

(52) U.S. Cl. CPC A61F 5/56

(57) ABSTRACT

The present disclosure relates to an anti-snoring device, and more specifically, to an anti-snoring device, which is inserted into the nasal cavity and comes into contact with the inner side of the nasal cavity. The anti-snoring device includes an outer wall portion having a cut-away area portion formed therein and a protrusion portion formed on the outer wall portion, so that the device can be flexibly and deeply inserted in accordance with the structure of the nasal cavity and can enhance the expandability of the nasal cavity. The present disclosure comprises: a body outer wall portion (201); a cut-away portion (203) formed in the body outer wall portion (201); and a protrusion portion (205) formed under the cut-away area portion (203) on the body outer wall portion (201).

### BACKGROUND OF THE DISCLOSURE

### Field of the Disclosure

The present disclosure relates to an anti-snoring device, and more specifically, to an anti-snoring device which is inserted into a nasal cavity and has a cut-away area portion disposed on an outer wall portion of the anti-snoring device coming into contact with an inner surface of the nasal cavity, and has a protrusion portion disposed on the outer wall portion, so it can be flexibly and deeply inserted in accordance with the structure of the nasal cavity and can enhance expandability of the nasal cavity.

### Related Art

In general, snoring, which is a slang term for obstructive breathing, has a relatively high incidence rate, and is considered as a disease with the development of airway physiology.

Such snoring occurs when a throat, through which air passes before entering an airway, becomes narrow while a human body breathes and the air may not easily enter and exit, and may be said to be one symptom that may indicate difficulty breathing during sleep.

In addition, when the air passage is completely blocked due to severe muscle relaxation during sleep or other causes such as severe obesity, air cannot flow into a lung at all, which is called sleep apnea.

When the sleep apnea condition continues, since a lung may not get fresh air, a brain that senses this condition wakes up our body and contracts muscles to open an air passage again, so breathing begins again after taking a deep breath.

Looking at the symptoms of the snoring and sleep apnea, a turbinate portion of a nasal cavity, whose inner surface is covered with mucous membrane has rich venous plexus, as it swells due to chills, a nasal passage is obstructed, so the amount of air entering a nose decreases, making it difficult to breathe.

If a person sleeps facing upward while the nasal passage is narrowed, when a tongue muscle sags inward of a throat, narrowing or obstructing the air passage, and air is inhaled through the narrow passage, he/she snores while a relaxed soft palate vibrates.

As such, when the sleep apnea occurs or the snoring is severe during the sleeping, a person may not breathe enough air. As a result, a person may get a good night's sleep, feels sleepy or has a heavy head and does not feel refreshed even after sleeping, has decreased concentration and judgment, and has a high risk of getting into a car accident due to drowsy driving, and furthermore, when this condition continues, oxygen deficiency increases the burden on the heart or lung, and causes serious complications such as high blood pressure, heart attack, and seizures.

### SUMMARY

The present disclosure provides an anti-snoring device capable of increasing a nasal cavity respiration volume by wearing on a nasal cavity to expand the nasal cavity, thereby preventing snoring.

In addition, the present disclosure provides a structure that can be flexibly and deeply inserted into the nasal cavity corresponding to the nasal cavity structure by disposing a cut-away area portion on an upper portion of an outer wall portion of an anti-snoring device.

The present disclosure provides a structure of nasal cavity expandability force of an anti-snoring device by providing a protrusion on an outer wall portion of the anti-snoring device to reinforce an outer wall portion.

The present disclosure provides a structure that is fixed when worn on the nasal cavity and does not easily separated by disposing a protrusion on an upper portion of an outer wall portion of the anti-snoring device.

The present disclosure is not limited to the above-mentioned effects, and other objects that are not mentioned may be obviously understood by those skilled in the art from the following description.

In an aspect, an anti-snoring device includes: a body outer wall portion (201); a cut-away area portion (203) formed on the body outer wall portion (201); and a protrusion (205) formed on the body outer wall portion (201), in which a support member (207) including a film is formed on the cut-away area portion (203) formed on the body outer wall portion (201) in a state smaller than a thickness or strength of the body outer wall portion (201).

An anti-snoring device includes: a body outer wall portion (201); a cut-away area portion (203) formed on the body outer wall portion (201); and a protrusion (205) provided under the cut-away area portion (203) formed in the body outer wall portion (201), in which a support member (207) including a film is formed on the cut-away area portion (203) formed on the body outer wall portion (201)

in a state smaller than a thickness or strength of the body outer wall portion (201).

The shape of the cut-away area portion (203) may be U-shaped or V-shaped.

A semi-circular protrusion (205) may be provided under the cut-away area portion (203) formed on the body outer wall portion (201) in an outward direction of the outer wall portion.

### Effects of the Invention

According to the present disclosure, the anti-snoring device can be deeply inserted into a nasal cavity corresponding to a nasal cavity structure.

In addition, according to the present disclosure, the anti-snoring device can be worn on the nasal cavity to expand the nasal cavity, thereby increasing a nasal cavity respiratory volume and preventing snoring.

In addition, according to the present disclosure, a protrusion can be formed on an upper portion of an outer wall portion of the anti-snoring device to provide a structure that expands a nasal cavity and is fixed when worn in the nasal cavity and does not easily separated.

In addition, by forming a protrusion on an upper portion of an outer side portion of an anti-snoring device, it is possible to improve an elastic support force of the outer wall portion of the anti-snoring device outward in a nasal cavity direction, improve expandability of a nasal cavity, and improve a fixation in the nasal cavity.

### BRIEF DESCRIPTION OF THE DRAWINGS

FIG. 1 is a perspective view of an anti-snoring device according to an embodiment of the present disclosure.

FIG. 2 is a front view of the anti-snoring device according to the embodiment of the present disclosure.

FIG. 3 is a side view of the anti-snoring device according to the embodiment of the present disclosure.

FIG. 4 is a view showing a cross section of a body outer wall portion (201) and a body inner wall portion (20) according to an embodiment of the present disclosure.

FIG. 5 is a view showing that the cross-sectional shape of FIG. 4 is implemented in one anti-snoring device.

FIG. 6 is a view showing that the cross-sectional shape of FIG. 4 is implemented in two anti-snoring devices.

FIG. 7 is a view showing that a runny nose leak prevention device (102) is provided on the outer wall portion of the anti-snoring device.

FIG. 8 is a perspective view of a support member (207) including a film formed in a cut-away area portion (203) formed on the outer wall portion in a state smaller than a thickness or strength of the outer wall portion of the anti-snoring device.

FIG. 9 is a front view of the support member (207) including the film formed in the cut-away area portion (203) formed on the outer wall portion in a state smaller than the thickness or strength of the outer wall portion of the anti-snoring device.

FIG. 10 is a plan view of the support member (207) including the film formed in the cut-away area portion (203) formed on the outer wall portion in a state smaller than the thickness or strength of the outer wall portion of the anti-snoring device.

FIG. 11 is a bottom view of the support member (207) including the film formed in the cut-away area portion (203) formed on the outer wall portion in a state smaller than the thickness or strength of the outer wall portion of the anti-snoring device.

FIG. 12 is a perspective view of the anti-snoring device according to another embodiment of the present disclosure.

FIG. 13 is a front view and a rear view of the anti-snoring device according to another embodiment of the present disclosure.

FIG. 14 is a left/right side view of the anti-snoring device according to another embodiment of the present disclosure.

FIG. 15 is a plan view of the anti-snoring device according to another embodiment of the present disclosure.

FIG. 16 is a bottom view of the anti-snoring device according to another embodiment of the present disclosure.

FIGS. 17 to 23 are a cross-sectional view of the anti-snoring device according to another embodiment of the present disclosure related to FIGS. 12 to 16.

FIG. 24 is an enlarged perspective view of the anti-snoring device of the present disclosure.

### DESCRIPTION OF EXEMPLARY EMBODIMENTS

Hereinafter, an embodiment according to the present disclosure will be described in detail with reference to the accompanying drawings.

However, since the accompanying drawings illustrate preferred embodiments of present disclosure, the scope of protection of present disclosure is not limited by these embodiments. In addition, even if the configuration necessary to implement the present disclosure is introduced in the prior art, or a matter that can be easily implemented by a person skilled in the art from the known technology, for example, a detailed description of a crown portion and the like will be omitted.

It is to be noted that in giving reference numerals to components of the accompanying drawings, the same components will be denoted by the same reference numerals even though they are illustrated in different drawings. In addition, in describing an embodiment of the present disclosure, a detailed description of substantially the same content will be omitted.

When it is mentioned that any component is “connected” or “coupled” to another component, it is to be understood that any component is directly connected or coupled to another component or is connected or coupled to another component with the other component interposed therebetween.

In addition, when it is described that a component is provided, formed, or constituted, it should be construed in substantially the same sense unless specifically defined.

In addition, in the present disclosure, the meaning of including is an open meaning that other components can be additionally included.

Terms such as an upper end and a lower end, an upper surface and a lower surface, or an upper portion and a lower portion are used in order to distinguish relative positions of components from each other. For example, for convenience, if the upper portion on the drawing is named an upper portion/upper side and the lower portion on the drawing is named a lower portion/lower side, in reality, the upper portion may be named as the lower portion without departing from the scope of the present disclosure, and the lower portion may be called the upper portion.

Hereinafter, an embodiment of the present disclosure will be described with reference to each drawing.

First, general matters related to present disclosure will be described.

A size of the anti-snoring device is manufactured in multiple sizes so that it can be used according to the size of male/female or a nasal cavity.

The anti-snoring device is generally used in pairs to correspond to a nose hole of a human body, but only one anti-snoring device may be used.

In addition, a connecting ring 50 for connecting each of the anti-snoring devices to each other is in contact with one lower side of each anti-snoring device. In this case, a width diameter 60 of an upper portion of the connecting ring reinforcement in contact with the anti-snoring device is 3 to 4 times or more than the connecting ring width 70, and the shape is an inverted triangle shape.

In the case of less than 3 times above, there is a problem in bearing capacity/durability, and if it exceeds 4 times, elasticity is affected.

Therefore, by differentiating the width, the bearing capacity of the anti-snoring device is improved when inserted/removed into the nasal cavity, and durability is improved by preventing breakage.

The anti-snoring device may lack a support force since silicon is a main material.

To compensate for this and to provide elasticity to the inside of the nasal cavity to expand the nasal cavity and to be fixed in the nasal cavity and not to be separated, a protrusion is provided on the outer wall portion.

In addition, by adding 1 to 5 parts by weight of silver powder to 100 parts by weight of silicone to produce the anti-snoring device, there is an antibacterial effect and/or an effect of eradicating bacteria.

When the amount of silver powder is less than 1 part by weight, the effect is insignificant, and when it exceeds 5 parts by weight, the practical benefit is insufficient in terms of cost or effectiveness.

In the present disclosure, a plurality of protrusions configured in each outer wall portion may be provided.

The anti-snoring device of the present disclosure is formed in a conical shape that narrows in diameter toward the upper portion so that it can be inserted deep into the nasal cavity, and the upper portion and the lower surface are open for smooth circulation of air.

Looking at the structure and shape of the silicone (double tube, outer wall portion/inner wall portion), the anti-snoring device is formed in a conical shape that gradually narrows in the direction of the upper portion so that it can be deeply inserted into the nasal cavity, and thus, has a structure that can prevent snoring with smooth air flow while playing the role of widening the nasal cavity located deep inside the nose during sleep breathing.

Silicon is formed in a double layer to form an outer wall and an inner wall, and a buffer action and a large through hole are formed during insertion to ensure smooth air communication and a good fit. Since the nasal cavity is formed in a conical shape that narrows when inserted, and thus, serves to widen the nasal cavity as it goes into the nose (inhale twice as much air during breathing), so it can prevent the noise of snoring and can be used as an auxiliary device for air communication even during breathing.

The ‘ U’-shaped space part in the above is an example, and it can be changed into various shapes such as a triangle and a jar shape, which falls within the scope of the present disclosure.

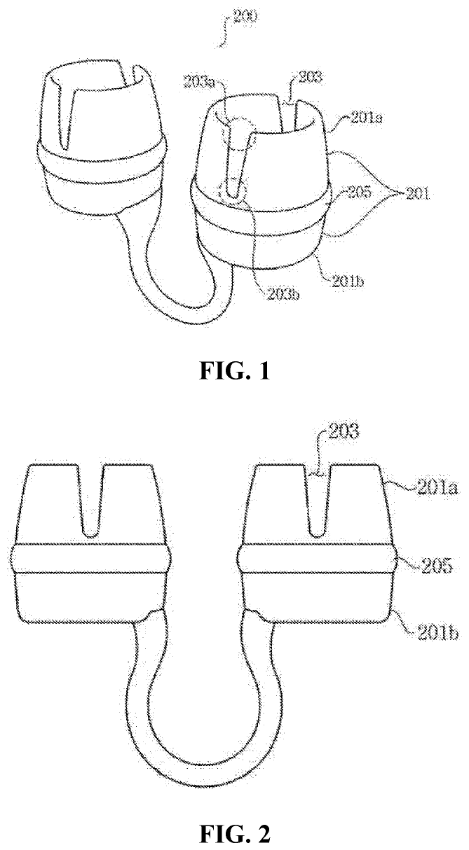

FIG. 1 is a perspective view of an anti-snoring device according to another embodiment of the present disclosure.

FIG. 2 is a front view of the anti-snoring device according to another embodiment of the present disclosure.

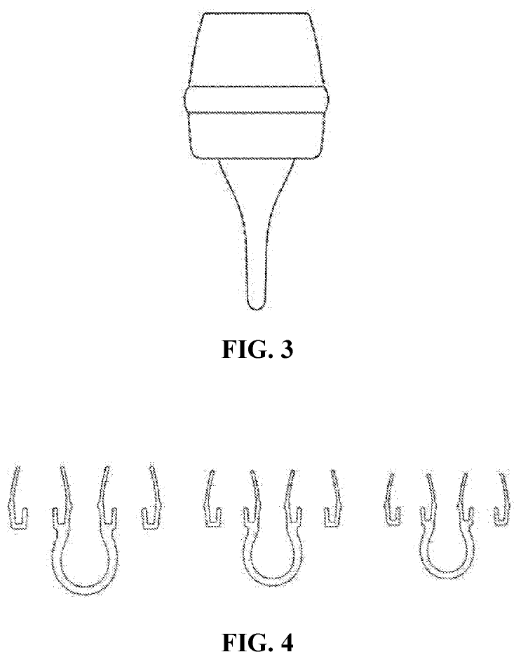

FIG. 3 is a side view of the anti-snoring device according to another embodiment of the present disclosure.

As shown in the figure, according to another embodiment, the anti-snoring device 200 includes a body outer wall portion 201 having an upper portion 201a and a lower portion 201b of the body outer wall portion, a cut-away area portion 203 formed by dividing the body outer wall portion 201 into a U or V type, and a protrusion 205 under the lower portion 203b of the cut-away area portion 203 formed in the body outer wall portion 201.

At this time, it is preferable to make a horizontal width of the upper portion 203a of the cut-away area portion 203 greater than or equal to that of the lower portion 203b, and in another embodiment, and vice versa (the divided horizontal width of the upper portion 203a may be smaller than that of the lower part 203b).

Also, a semi-circular protrusion 205 in an outer direction of the outer wall portion is preferably provided under the lower portion 203b of the cut-away area portion 203 formed in the body outer wall portion 201, and the protrusion shape may be an oval, a round type, or the like.

In the above, it is preferable that a diameter of the upper portion of the body outer wall portion (not shown) is smaller than that of the outer wall portion (not shown) in which the protrusion 205 is formed. This is to make it easier to insert the anti-snoring device into the nose. In addition, the reason for forming the cut-away area portion 203 in the upper portion of the outer wall portion 201a is that it expands the nasal cavity by putting elasticity outward when inserted into the nose, and is easily deeply inserted into the nasal cavity corresponding to the structure of the nasal cavity with flexibility.

The reason for forming the cut-away area portion formed in the body outer wall portion 201 in a U or V type is that the horizontal width of the upper portion 203a of the cut-away area portion 203 is greater than that of the lower portion 203b, and therefore, when inserted into the nose, the insertion may be deeply made by deforming the shape of the upper portion of the outer wall portion corresponding to the structure of the nasal cavity based on adequate flexibility.

On the other hand, if the focus is on nasal cavity expansion, the horizontal width of the upper portion 203a of the cut-away area portion 203 is equal to the width of the lower portion 203b, or the divided horizontal width of the upper portion 203a may be made smaller than the width of the lower portion 203b.

The reason why the protrusion 205 formed under the lower portion 203b the cut-away area portion 203 of the body outer wall portion 201 expands the nasal cavity by reinforcing and supporting the body outer wall portion, and the protrusion also has a round shape (semi-circular shape, oval shape, etc.) is that when an anti-snoring device is inserted into the nasal cavity, it is inserted flexibly and smoothly corresponding to the nasal cavity structure.

FIG. 4 is a view showing a cross section of the body outer wall portion 201 and a body inner wall portion 20 according to another embodiment of the present disclosure. As shown in the drawing, the semi-circular protrusion 205 is formed under the cut-away area portion of the body.

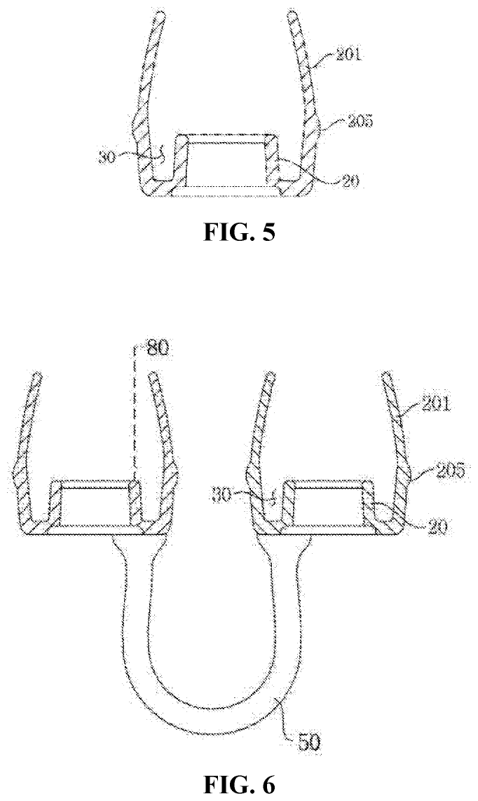

FIG. 5 is a view showing that the cross-sectional shape of FIG. 4 is implemented in one anti-snoring device.

FIG. 6 is a view showing that the cross-sectional shape of FIG. 4 is implemented in two anti-snoring devices.

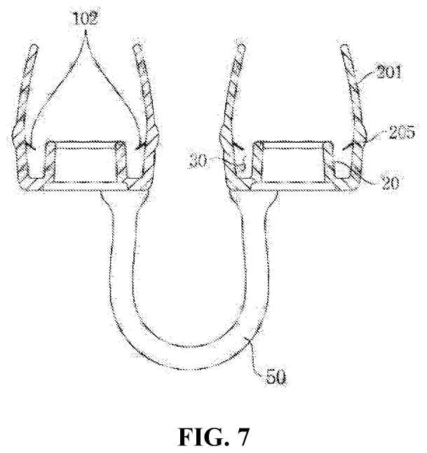

FIG. 7 is a view showing that a runny nose leak prevention device 102 is provided on the outer wall portion of the anti-snoring device.

Also, another embodiment of the present disclosure will be described.

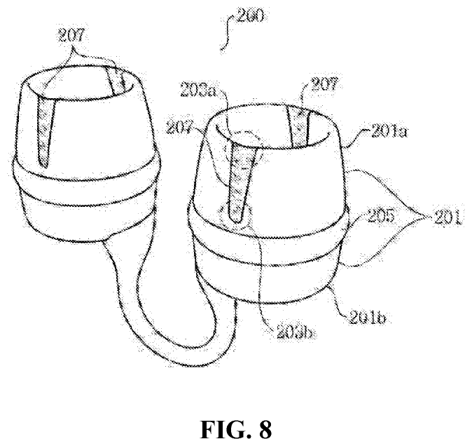

FIG. 8 is a perspective view of a support member 207 including a film formed in a cut-away area portion 203 formed in the outer wall portion in a state smaller than a thickness or strength of the outer wall portion of the anti-snoring device.

FIG. 9 is a front view of the support member 207 including the film formed in the cut-away area portion 203 formed in the outer wall portion in a state smaller than the thickness or strength of the outer wall portion of the anti-snoring device.

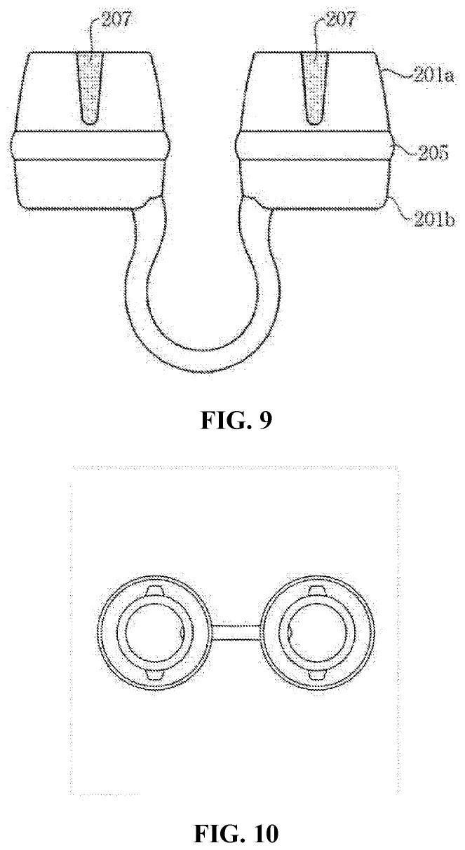

FIG. 10 is a plan view of the support member 207 including the film formed in the cut-away area portion 203 formed in the outer wall portion in a state smaller than the thickness or strength of the outer wall portion of the anti-snoring device.

FIG. 11 is a bottom view of the support member 207 including the film formed in the cut-away area portion 203 formed in the outer wall portion in a state smaller than the thickness or strength of the outer wall portion of the anti-snoring device.

As shown in FIG. 1, in the state where the cut-away area portion 203 formed on the body outer wall portion 201 is smaller than the thickness or strength of the body outer wall portion 201 rather than the just floating state, by forming the support member 207 including the film on the cut-away area portion 203 in the state of being in contact with the outer wall portion 201, the open outer wall portions 201 on both sides are connected to each other, and thus, the support force of the outer wall portion is improved when the body is inserted into the nasal cavity.

The thickness of the support member should be in the range of 40 to 60% of the thickness of the body outer wall portion (e.g., 0.4 to 0.5 mm). In this case, when the thickness of the support member is less than 40%, the purpose of supplementing the support force of the body outer wall portion 201 is not substantially affected, and when it exceeds 60%, the flexibility of the outer wall portion 201 is reduced, and thus, the outer wall portion 201 becomes difficult to insert into the nasal cavity.

In other words, the body outer wall portion is inserted into and in contact with the nasal cavity, and serves to expand the nasal cavity, so the outer wall portion should not be too flexible and should not be too hard.

TABLE 1

| Thickness | | |
|---|---|---|
| of support | | |
| member/Thickness | | |
| of outer wall | Support force of outer | Flexibility of outer wall |
| portion (%) | wall portion | portion |
| 30% or less | Same as without support | Same as without support |
| | member | member |
| 40% | Good (Sharply improved | Substantially the same |
| | support force) | as above |
| 60% | Same as above | Same as above |
| 70% | Same as above | Bad (Sharply reduced |
| | | flexibility) |

In the present disclosure, the body and support member materials include, as an example, silicone, a synthetic resin material, or a synthetic material. As described above, the present disclosure relates to an anti-snoring device, and more specifically, to an anti-snoring device which is inserted into a nasal cavity and has a cut-away area portion disposed on an outer wall portion of the anti-snoring device coming into contact with an inner surface of the nasal cavity, and has a protrusion portion disposed on the outer wall portion, so it can be flexibly and deeply inserted in accordance with the structure of the nasal cavity and can enhance expandability of the nasal cavity. In addition, the present disclosure relates to a device for improving elastic support force of the outer wall portion of the anti-snoring device, expendability of nasal cavity, and fixation in the nasal cavity by forming the protrusion on the upper portion of the outer side portion of the anti-snoring device inserted into the nasal cavity and coming into contact with the inner surface of the nasal cavity.

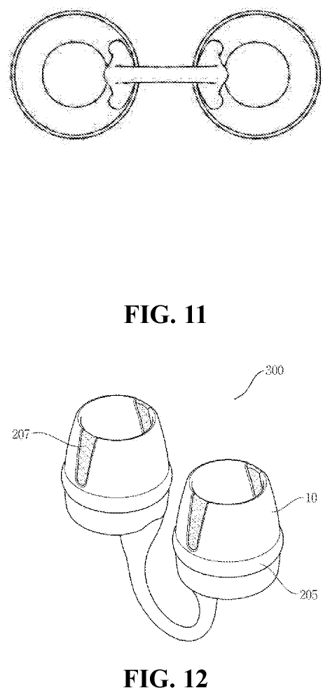

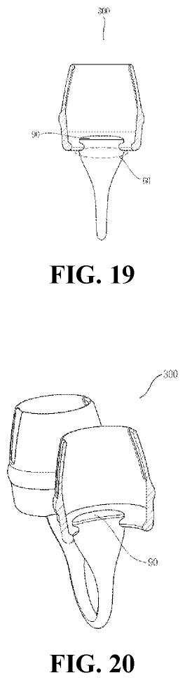

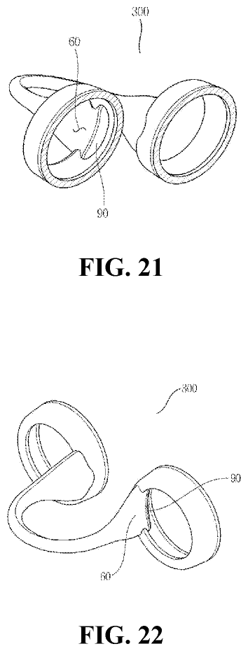

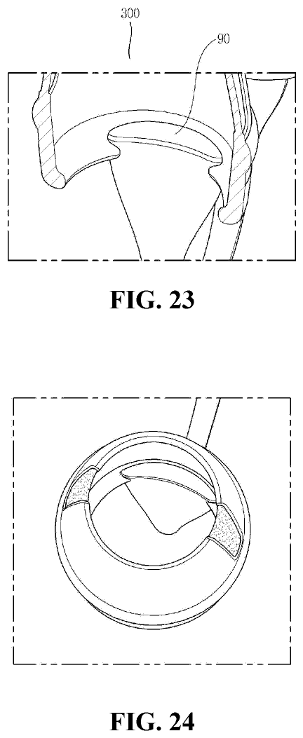

In addition, a snoring prevention device 300 according to another embodiment of the present disclosure will be described. [FIGS. 12 to 24]

Hereinafter, it will be described with reference to each drawing.

First, in describing this embodiment, reference numerals having the same purpose as those of the above-described embodiment and its drawings (especially FIGS. 1, 2, 5, 8, and 9, etc.) are used together.

FIGS. 12 to 16 are views showing respective views (a perspective view, a front view, a rear view, a side view, a top view, and a bottom view) of an anti-snoring device according to another embodiment of the present disclosure.

FIG. 12 is a perspective view of the anti-snoring device according to another embodiment of the present disclosure.

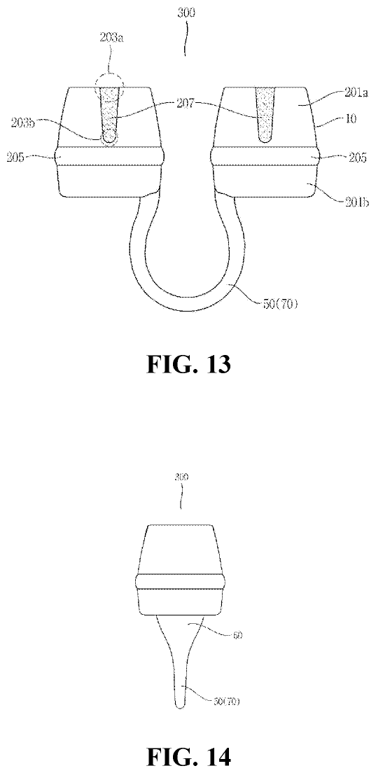

FIG. 13 is a front view and a rear view of the anti-snoring device according to another embodiment of the present disclosure.

FIG. 14 is a left/right side view of the anti-snoring device according to another embodiment of the present disclosure.

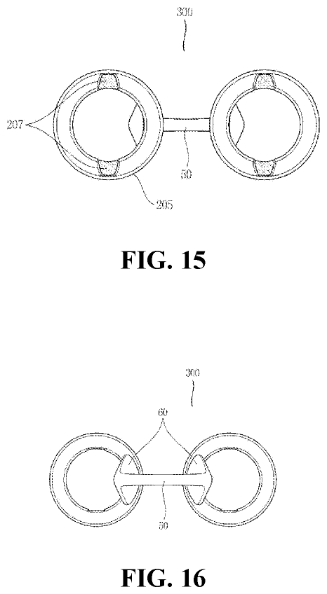

FIG. 15 is a plan view of the anti-snoring device according to another embodiment of the present disclosure.

FIG. 16 is a bottom view of the anti-snoring device according to the embodiment of the present disclosure

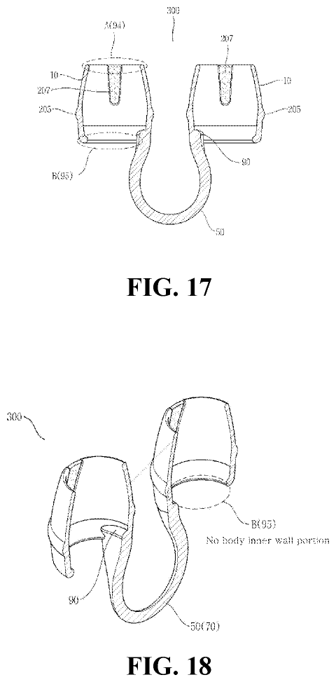

FIGS. 17 to 23 are views showing cross-sectional views of the anti-snoring device according to another embodiment of the present disclosure related to FIGS. 12 to 16. FIG. 24 is an enlarged perspective view showing the anti-snoring device of the present disclosure. As shown in the drawing,

the anti-snoring device includes a body outer wall portion 201; a cut-away area portion (203) formed on the body outer wall portion 201; and a protrusion 205 formed on the body outer wall portion 201.

Alternatively,

the anti-snoring device includes the body outer wall portion 201; the cut-away area portion 203 formed on the body outer wall portion 201; and the protrusion 205 under the cut-away area portion 203 formed on the body outer wall portion 201, and does not basically includes the body inner wall portion 20 according to the embodiment. As a result, air communication during inhalation or exhalation was improved through the anti-snoring device inserted into the nasal cavity, and in particular, it was possible to prevent noise caused by vibration of the inner wall of the body during exhalation (when air is discharged).

On the other hand, in order to supplement the support force of the anti-snoring device since there is no body inner wall portion as described above, a body reinforcement portion 90 is provided inside the lower portion 201b of the body outer wall portion.

In other words, a connecting ring 50 is in contact with one side of the lower portion of the body outer wall portion 201b, and a horizontal width 70 of a middle portion of the connecting ring becomes larger 60 in order to prevent breakage during attachment and detachment and to increase for support force as it approaches one side of the lower portion of the body outer wall portion, and a body reinforcement portion 90 is provided inside the lower portion of the body outer wall portion 201b in contact with (connected with) an end portion 60 of the increased horizontal width. Although the reinforcement portion 90 is expressed as a body reinforcement portion, it is to reinforce the connecting ring 50 as well as the body of the anti-snoring device.

An opening width (A) 94 of an upper end portion (portion located inside the nasal cavity) of the anti-snoring device in the absence of the inner wall of the body of the embodiment is generally smaller than an opening width (B) 95 of a lower end portion of the anti-snoring device It is common to make it smaller.

On the other hand, if necessary, the opening width (A) 94 may be equal to or similar to the opening width (B) 95 of the lower end portion of the anti-snoring device.

On the other hand, when the opening of the lower end portion of the anti-snoring device is thickened for strength of the lower portion of the body outer wall portion, the opening width (A) 94 may be larger than the opening width (B) 95 of the lower end portion of the anti-snoring device.

The spirit of the present disclosure has been illustratively described hereinabove. It will be appreciated by a person of ordinary skill in the art that various modifications and alterations may be made without departing from the essential characteristics of the present disclosure. Therefore, the embodiments implemented in the present disclosure are not intended to limit the technical idea of the present disclosure, but to explain, and the scope of the technical idea of the present disclosure is not limited by this embodiment. The scope of the present disclosure should be interpreted by the following claims and it should be interpreted that all spirits equivalent to the following claims fall within the scope of the present disclosure.

### INDUSTRIAL AVAILABILITY

The present disclosure can also be used for an anti-snoring device, a device for preventing fine dust containing dust, and can be for preventing foreign substances or viruses from entering a human body from the outside.

1. An anti-snoring device, comprising:
a body outer wall portion (201);
a cut-away area portion (203) formed on the body outer wall portion (201); and
a protrusion (205) formed on the body outer wall portion (201),
wherein a support member (207) including a film is formed on the cut-away area portion (203) formed on the body outer wall portion (201) in a state smaller than a thickness or strength of the body outer wall portion (201).

2. An anti-snoring device, comprising:
a body outer wall portion (201);
a cut-away area portion (203) formed on the body outer wall portion (201); and
a protrusion (205) provided under the cut-away area portion (203) formed in the body outer wall portion (201),
wherein a support member (207) including a film is formed on the cut-away area portion (203) formed on the body outer wall portion (201) in a state smaller than a thickness or strength of the body outer wall portion (201).

3. The anti-snoring device of claim 1, wherein the shape of the cut-away area portion (203) is U-shaped or V-shaped.

4. The anti-snoring device of claim 2, wherein the shape of the cut-away area portion (203) is U-shaped or V-shaped.

5. The anti-snoring device of claim 1, wherein a semi-circular protrusion (205) is provided under the cut-away area portion (203) formed on the body outer wall portion (201) in an outward direction of the outer wall portion.

6. The anti-snoring device of claim 2, wherein a semi-circular protrusion (205) is provided under the cut-away area portion (203) formed on the body outer wall portion (201) in an outward direction of the outer wall portion.

7. The anti-snoring device of claim 1, wherein the thickness of the support member is in a range of 40-60% of the thickness of the body outer wall portion.

8. The anti-snoring device of claim 2, wherein the thickness of the support member is in a range of 40-60% of the thickness of the body outer wall portion.

9. The anti-snoring device of claim 1, wherein a body reinforcement portion (90) is provided inside the body outer wall portion (201b).

10. The anti-snoring device of claim 2, wherein a body reinforcement portion (90) is provided inside the body outer wall portion (201b).

11. The anti-snoring device of claim 1, wherein a connecting ring (50) is in contact with one side of the lower portion of the body outer wall portion (201b), and a horizontal width (70) of the connecting ring becomes larger (60) as it approaches one side of the lower portion of the body outer wall portion, and a body reinforcement portion (90) is provided inside the lower portion of the body outer wall portion (201b) in contact with an end portion (60) of the increased horizontal width.

12. The anti-snoring device of claim 2, wherein a connecting ring (50) is in contact with one side of the lower portion of the body outer wall portion (201b), and a horizontal width (70) of the connecting ring becomes larger (60) as it approaches one side of the lower portion of the body outer wall portion, and a body reinforcement portion (90) is provided inside the lower portion of the body outer wall portion (201b) in contact with an end portion (60) of the increased horizontal width.
